SEQUENCE LISTING (2 sequences)

SEQ ID NO: 1 - DNA, 3822 bp, coronavirus 2 isolate Wuhan-Hu-1
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 2 - protein, 1273 aa, coronavirus 2 isolate Wuhan-Hu-1
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

(19) United States
(12) Patent Application Publication
(10) Pub. No.: US 2023/0149534 A1
(43) Pub. Date: May 18, 2023

(54) HAPTENIZED CORONAVIRUS SPIKE PROTEINS

(71) Applicant: BIOVAXYS INC., New York, NY (US)

(72) Inventors: David Berd, Jenkintown, PA (US); James Passin, New York, NY (US)

(21) Appl. No.: 17/906,784

(22) PCT Filed: Mar. 19, 2021
(86) PCT No.: PCT/US2021/023310

§ 371 Date: Sep. 20, 2022

Related U.S. Application Data
(60) Provisional application No. 62/992,722, filed on Mar. 20, 2020.

(51) Int. Cl. A61K 39/215; A61K 39/385

(52) U.S. Cl. CPC A61K 39/215; A61K 39/385; A61K 2039/545

(57) ABSTRACT

The disclosure provides an immunogenic composition comprising an haptenized Spike protein (S protein) or fragment thereof from a coronavirus and at least one pharmaceutically acceptable carrier, wherein the coronavirus comprising severe acute respiratory syndrome coronavirus 2 (SARS-CoV-2). Further disclosed are methods of using the haptenized S proteins from coronavirus or immunizing a subject against coronavirus infection.

### FIELD OF THE INVENTION

The invention described herein relates generally to a haptenized Spike protein (S protein) from coronavirus and methods of immunizing human subjects with haptenized Spike proteins.

### BACKGROUND OF THE INVENTION

Coronaviruses are plus-strand RNA viruses that cause disease in animals and humans. A novel zoonotic coronavirus outbreak started in Wuhan, China in 2019. This pandemic disease has now been defined as novel coronavirus disease 2019 (Covid-19), and is sustained by severe acute respiratory syndrome coronavirus 2 (SARS-CoV-2).

With such a new disease, many unresolved issues remain, even including the mode of transmission of the causal agent of Covid-19. The major risk for transmission of the SARS-CoV-2 virus is apparently by droplet exposure and close personal contact; therefore, strategies to reduce transmission of this coronavirus should parallel or mimic those used to limit other respiratory tract infections, i.e. reduce immediate contact and use barrier precautions against exposure to droplets. However, because the incubation period lasts from seven to fourteen days and non-specific initial symptoms are similar to those of other respiratory tract infections, such as influenza, the greatest risk for spread of Covid-19 is undetected cases. Thus, a need exists for alternative prophylactic strategies or therapies to treat or prevent coronavirus infections, such as those found in humans (e.g. SARS-CoV-2 infections resulting in Covid-19). For example, a need exists for identifying and developing immunogenic and vaccine compositions against coronavirus infections that can elicit a protective immune response. Furthermore, immunogenic compositions and vaccine formulations are needed that can be delivered directly to or in close proximity to the site of infection to maximize therapeutic effectiveness.

Haptenized proteins have been widely used to provide defined epitopes for the measurement of antibody titers and affinities. While some protein-haptenation is thought to do little more than create epitopes for B cell recognition, others have shown that certain haptenized proteins can induce adaptive immune responses under conditions where native proteins fail to induce such responses; thus, haptenation does more than simply create epitopes for antigen receptor recognition (Palm, Proc. Natl. Acad. Sci. USA (2000) 106:4782). The present invention meets such needs and further provides other related advantages. The present invention provides for haptenized immunogens for coronavirus and meets the need for immunogenic compositions and vaccine formulations that can be delivered directly or in close proximity to the site of infection to maximize a protective immune response in a human subject.

### SUMMARY OF THE INVENTION

Embodiments disclosed herein are immunogenic compositions or vaccines comprising haptenized S proteins from coronavirus, including severe acute respiratory syndrome coronavirus 2 (SARS-CoV-2), and methods of immunizing a human subject with the immunogenic composition or vaccine.

### BRIEF DESCRIPTION OF THE DRAWINGS

The foregoing summary, as well as the following detailed description of the invention, will be better understood when read in conjunction with the appended drawings.

FIG. 1 shows the anti-spike protein antibody respond following subcutaneous administration of BVX-0320 in CF-1 mice.

FIG. 2 shows the T cell (gamma interferon) response to BVX-0320.

### DETAILED DESCRIPTION OF THE INVENTION

Unless defined otherwise, all technical and scientific terms used herein have the same meaning as is commonly understood by one of skill in the art to which this invention belongs. All patents and publications referred to herein are incorporated by reference in their entireties.

### Definitions

An “immunogenic composition” or “vaccine” as used herein refers to any one or more compounds or agents or immunogens capable of priming, potentiating, activating, eliciting, stimulating, augmenting, boosting, amplifying, or enhancing an adaptive (specific) immune response, which may be cellular (T cell) or humoral (B cell), or a combination thereof. Preferably, the adaptive immune response is protective, which may include neutralization of a coronavirus (decreasing or eliminating virus infectivity). A representative example of an immunogen is a viral antigen (such as one or more coronavirus antigens). In the present description, any concentration range, percentage range, ratio range, or integer range is understood to include the value of any integer within the recited range and, when appropriate, fractions thereof (such as one tenth and one hundredth of an integer, etc.), unless otherwise indicated.

As used herein, “about” or “comprising essentially of” means ±10%. As used herein, the use of an indefinite article, such as “a” or “an,” should be understood to refer to the singular and the plural of a noun or noun phrase (i.e. meaning “one or more” or “at least one” of the enumerated elements or components). The use of the alternative (e.g. “or”) should be understood to mean either one, both or any combination thereof of the alternatives. In addition, it should be understood that the individual compounds, or groups of compounds, derived from the various combinations of the sequences, structures, and substituents described herein, are disclosed by the present application to the same extent as if each compound or group of compounds was set forth individually. Thus, selection of particular sequences, structures, or substituents is within the scope of the present invention.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g. the weight, age and gender of the subject), the severity of the disease condition, the manner of administration, etc. which can readily be determined by one of ordinary skill in the art. The term also applies to a dose that will induce a particular response in target cells (e.g. the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

The term “Immunogenicity” means the ability of an S protein immunogen to evoke an immune response directed to the coronavirus. Whether a haptenized S protein preparation is immunogenic can be tested by, for instance, a DTH-assay or an in vivo assay in an experimental animal model.

A “therapeutic effect” as that term is used herein, encompasses a therapeutic benefit and/or a prophylactic benefit. A prophylactic effect includes delaying or eliminating the appearance of a coronavirus infection, delaying or eliminating the onset of symptoms of a coronavirus infection, slowing, halting, or reversing the progression of a coronavirus infection, or any combination thereof.

“Pharmaceutically acceptable carrier” or “pharmaceutically acceptable excipient” is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the invention is contemplated. Additional active pharmaceutical ingredients, such as other drugs, can also be incorporated into the described compositions and methods.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

As used herein, “isotype” refers to the antibody class (e.g. IgM or IgG1) that is encoded by the heavy chain constant region genes. In mammals, there are five antibody isotypes: IgA, IgD, IgG, IgM and IgE. In humans, there are four subclasses of the IgG isotype: IgG1, IgG2, IgG3 and IgG4, and two subclasses of the IgA isotype: IgA1 and IgA2.

The terms “sequence identity” and “sequence percent identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. Suitable programs to determine percent sequence identity include for example the BLAST suite of programs available from the U.S. Government's National Center for Biotechnology Information BLAST web site. Comparisons between two sequences can be carried using either the BLASTN or BLASTP algorithm. BLASTN is used to compare nucleic acid sequences, while BLASTP is used to compare amino acid sequences. ALIGN, ALIGN-2 (Genentech, South San Francisco, Calif.) or MegAlign, available from DNASTAR, are additional publicly available software programs that can be used to align sequences. One skilled in the art can determine appropriate parameters for maximal alignment by particular alignment software. In certain embodiments, the default parameters of the alignment software are used.

For the avoidance of doubt, it is intended herein that particular features (for example integers, characteristics, values, uses, diseases, formulae, compounds or groups) described in conjunction with a particular aspect, embodiment or example of the invention are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any disclosed embodiments. The invention extends to any novel one, or novel combination, of the features disclosed in this specification (including any accompanying claims, abstract and drawings), or to any novel one, or any novel combination, of the steps of any method or process so disclosed.

### Methods of Preparing Haptenized S Protein

The preparation of autologous vaccines using a single hapten reagent are known to the skilled artisan. In an embodiment, the invention provides for an haptenized S protein coronavirus vaccine, such a vaccine may be prepared comprising recombinantly producing an S protein or fragment thereof, and haptenizing with a haptenization reagent.

The step of haptenizing may be performed by modifying recombinant S protein with DNP by a 30-minute incubation with the haptenization reagent 2,4-difluoronitrobenzene (DNFB). The haptenized S protein is washed with HBSS. Preferred haptenization reagents may target the ϵ-amino group of an amino acid.

In some embodiments, the haptenization reagent is selected from trinitrochlorobenzene (TNCB), 2,4-difluoronitrobenzene (DNFB), N-iodoacetyl-N′-(5-sulfonic-1-naphthyl)ethylenediamine (AED), sulfanilic acid (SA), trinitrophenol (TNP), 2,4,6-trinitrobenzenesulfonic acid (TNBS) and combinations thereof.

A haptenized S protein immunogen (and corresponding immunogenic epitopes) and fragments, and variants thereof may be produced synthetically or recombinantly. A coronavirus S protein fragment that contains an epitope that induces an immune response against coronavirus may be synthesized by standard chemical methods, including synthesis by automated procedure. In general, immunogenic peptides are synthesized based on the standard solid-phase Fmoc protection strategy with HATU as the coupling agent. The immunogenic peptide is cleaved from the solid-phase resin with trifluoroacetic acid containing appropriate scavengers, which also deprotects side chain functional groups. The crude immunogenic peptide may be further purified using preparative reverse phase chromatography. Other purification methods, such as partition chromatography, gel filtration, gel electrophoresis, or ion-exchange chromatography may be used. Other synthesis techniques known in the art may be employed to produce similar immunogenic peptides, such as the tBoc protection strategy, use of different coupling reagents, and the like. In addition, any naturally occurring amino acid or derivative thereof may be used, including D-amino acids or L-amino acids, and combinations thereof. In certain embodiments, a synthetic S protein immunogen has an amino acid sequence that is identical to, or at least 85% identical (which includes at least 90% or 95% or any percent in between 85% and 100%) to SEQ ID NO:2.

As described herein, the haptenized S protein immunogens may be recombinant, wherein desired S protein immunogens are individually or in combination expressed from a polynucleotide that is operably linked to an expression control sequence (e.g. a promoter) in a nucleic acid expression construct. In certain embodiments, a recombinant S protein antigen will comprise an amino acid sequence that is identical to, or at least 85% identical (which includes at least 90% or 95% or any percent in between 80% and 100%) to SEQ ID NO:2. In another embodiment, a recombinant S protein immunogen consists of an amino acid sequence as set forth in SEQ ID NO:2. In other embodiments, recombinant S protein immunogens and variants thereof are fragments of SEQ ID NO:2. In some embodiments, the variants are SARS-CoV-2 Spike protein variants found in different strains of SARS-CoV-2. Exemplary variants of the Spike protein from these different strains are set forth in Table 1. Variants include, but are not limited to, Spike proteins from the B.1.1.7 strain, B.1.351 strain, P.1 strain, CAL 20 strain or any combination thereof.

TABLE 1
List of amino acid positions and relative amino acid
changes in the different variants in the Spike protein
with respect to the ancestral Wuhan strain
Spike protein (SEQ ID NO: 2).

| protein | position | Wuhan | B1.1.7 | B1.351 | P.1 | CAL.20 |
|---|---|---|---|---|---|---|
| S | 13 | S | | | | I |
| S | 18 | L | | F | F | |
| S | 20 | T | | | N | |
| S | 26 | P | | | S | |
| S | 69 | H | Del | | | |
| S | 70 | V | Del | | | |
| S | 80 | D | | A | | |
| S | 138 | D | | | Y | |
| S | 145 | Y | Del | | | |
| S | 152 | W | | | | C |
| S | 190 | R | | | S | |
| S | 215 | D | | G/H | | |
| S | 241 | L | | Del | | |
| S | 242 | L | | Del | | |
| S | 243 | A | | Del | | |
| S | 417 | K | | N | T | |
| S | 452 | L | | | | R |
| S | 484 | E | | K | K | |
| S | 501 | N | Y | Y | Y | |
| S | 570 | A | D | | | |
| S | 614 | D | G | G | G | G |
| S | 655 | H | | | Y | |
| S | 681 | P | H | | | |
| S | 701 | A | | V | | |
| S | 716 | T | I | | | |
| S | 938 | L | | | | F |
| S | 982 | S | A | | | |
| S | 1027 | T | | | I | |
| S | 1118 | D | H | | | |
| S | 1176 | V | | | F | |
| S | 1191 | K | | | | N |

In one embodiment, the S proteins are haptenized. For purposes of the present invention, virtually any small protein or other small molecule that fails to induce an immune response when administered alone, may function as a hapten. A variety of haptens of quite different chemical structure have been shown to induce similar types of immune response, e.g. TNP (Kempkes, J. Immunol. (1991) 147:2467); phosphorylcholine (Jang, Eur. J. Immunol. (1991) 21:1303); nickel (Pistoor, J. Invest. Dermatol. (1995) 105:92) and arsenate (Nalefski, J. Immunol. (1993) 150:3806). Conjugation of a hapten to a cell to elicit an immune response may preferably be accomplished by conjugation via ε-amino groups of lysine or —COOH groups. This group of haptens include a number of chemically diverse compounds: dinitrophenyl, trinitrophenyl, N-iodoacetyl-N′-(5-sulfonic 1-naphthyl) ethylene diamine, trinitrobenzene-sulfonic acid, dinitrobenzene sulfonic acid, fluorescein isothiocyanate, arsenic acid benzene isothiocyanate, and dinitrobenzene-S-mustard (Nahas, Cellular Immunol. (1980) 54:241). Once armed with the present disclosure, skilled artisans would be able to choose haptens for use in the present invention.

In general, haptens include a “recognition group” which is the group that interacts with an antibody. The recognition group is irreversibly associated with the hapten reactive group. Thus, when the hapten reactive group is conjugated to a functional group on the target molecule, the hapten recognition group is available for binding with antibody. Examples of different hapten recognition groups include without limitation to dinitriophenyl, trinitrophenyl, fluorescein, other aromatics, phosphorylcholine, peptides, advanced glycosylation endproducts (AGE), carbohydrates, etc.

Haptens also include a functional group for conjugation to a substituent on an amino acid side chain of a protein or polypeptide. Amino acid side chain groups that can be conjugated to hapten include, e.g. free carboxylic acid groups in the aspartic acid or glutamic acid; the ε-amino group of lysine; the thiol moiety of cysteine; the hydroxyl group of serine or tyrosine; the imidazole moiety of histidine; or the aryl groups of tryptophan, tyrosine, or phenylalanine. Hapten functional groups capable of reacting with specific amino acid side chains are described as follows.

Functional groups reactive with primary amines. Hapten reactive groups that would form a covalent bond with primary amines present on amino acid side chains would include, but not be limited to, acid chlorides, anhydrides, reactive esters, α,β-unsaturated ketones, imidoesters, and halonitrobenzenes. Various reactive esters with the capability of reacting with nucleophilic groups such as primary amines are available commercially. Functional groups reactive with carboxylic acids. Carboxylic acids in the presence of carbodiimides, such as EDC, can be activated, allowing for interaction with various nucleophiles, including primary and secondary amines. Alkylation of carboxylic acids to form stable esters can be achieved by interaction with sulfur or nitrogen mustards, or haptens containing either an alkyl or aryl aziridine moiety.

Functional groups reactive with aromatic groups. Interaction of the aromatic moieties associated with certain amino acids can be accomplished by photoactivation of aryl diazonium compound in the presence of the protein or peptide. Thus, modification of the aryl side chains of histidine, tryptophan, tyrosine, and phenylalanine, particularly histidine and tryptophan, can be achieved by the use of such a reactive functionality.

Functional groups reactive with sulfhydryl groups. There are several reactive groups that can be coupled to sulfhydryl groups present on the side chains of amino acids. Haptens containing an α,β-unsaturated ketone or ester moiety, such as maleimide, provide a reactive functionality that can interact with sulfhydryl as well as amino groups. In addition, a reactive disulfide group, such as 2-pyridyldithio group or a 5,5′-dithio-bis-(2-nitrobenzoic acid) group is also applicable. Some examples of reagents containing reactive disulfide bonds include N-succinimidly 3-(2-pyridyl-dithio) propionate (Carlsson, Biochem J. (1978) 173:723-737), sodium S-4-succinimidyloxycarbonyl-alpha-methylbenzyl-thiosulfate, and 4 succinimidyloxycarbonyl-alpha-methyl-(2-pyridyldithio)-toluene. Some examples of reagents comprising reactive groups having a double bond that reacts with a thiol group include succinimidyl 4-(N-maleimidomethyl)cyclohexahe-1-carboxylate and succinimidyl m-maleimidobenzoate.

Other functional molecules include succinimidyl 3-(maleimido)-propionate, sulfosuccinimidyl 4-(p-maleimido-phenyl)butyrate, sulfo-succinimidyl-4-(N-maleimidomethyl-cyclohexane)-1-carboxylate, maleimidobenzolyl-N-hydroxy-succinimide ester.

Any hapten or combination of different haptens can be used in the compositions of the invention. For example in one embodiment, the same hapten recognition group is coupled to different amino acids through different functional groups on the S protein. For example, the reagents dinitrobenzene sulfonic acid, dinitro phenyldiazonium, and dinitrobenzene S mustard, all form the dinitrophenyl hapten coupled to amino groups, aromatic groups, and carboxylic acid groups, respectively. Similarly, an arsonic acid hapten can be coupled by reacting arsonic acid benzene isothiocyanate to amino groups or azobenzenearsonate to aromatic groups.

### Immunogenic Compositions & Vaccines

Immunogenic compositions or vaccines as described herein useful for treating and/or preventing a coronavirus infection comprises immunogenic haptenized coronavirus polypeptides, such as S protein, fragments, and variants thereof, and also includes a fusion of a coronavirus immunogen to other peptides or polypeptides (e.g. a hydrophobic amino acid sequence or a histidine tag or a non-S protein coronavirus polypeptide or fragment thereof) or other modifications (e.g. glycosylation). In certain embodiments, the immunogenic S polypeptides may comprise any portion of an S protein that has an epitope capable of eliciting a protective immune response (e.g. eliciting production of a neutralizing antibody and/or stimulating a cell-mediated immune response) against a coronavirus infection. Immunogenic polypeptides as described herein may be arranged, combined, or fused in a linear form, and each immunogen may or may not be reiterated, wherein the reiteration may occur once or multiple times, and may be located at the N-terminus, C-terminus, or internal to a linear sequence of immunogenic S or other coronavirus polypeptide immunogens. In addition, a plurality of different coronavirus immunogenic polypeptides (e.g. other S proteins from multiple coronavirus species, N proteins, M proteins, or other coronavirus polypeptides, and variants or fragments thereof) can be selected and mixed or combined into a cocktail composition to provide a multivalent vaccine for use in eliciting a protective immune response without a harmful or otherwise unwanted associated immune responses or side effects. Also provided herein are methods for producing a haptenized synthetic or recombinant multivalent coronavirus polypeptide immunogens, including fusion proteins. For example, host cells containing an S protein immunogen-encoding nucleic acid expression construct may be cultured to produce the recombinant S protein immunogen, or variants thereof (e.g. deletion mutants or S polypeptide fragments lacking a C-terminal transmembrane domain). Also contemplated are methods for treating or preventing coronavirus infections or eliciting an immune response using an S protein immunogen or variant thereof, or a combination of polypeptides (including fusion proteins).

Coronavirus has a positive-sense, non-segmented, single-stranded RNA genome, which encodes at least 18 viral proteins (such as non-structural proteins (NSP) 1-13, structural proteins E, M, N, S), and an RNA-dependent RNA polymerase). Coronavirus has three major surface glycoproteins (designated S, E, and M), and some coronaviruses have another surface glycoprotein referred to as hemagglutinin esterase (HE), which is not found in the SARS virus, the N (nucleocapsid) protein is a basic phosphoprotein, which is generally associated with the genome and has been reported to be antigenic (Holmes, Fields Virology, Chapter 34, 2013). The S (spike) protein, a major antigen of coronavirus, has two domains: SI, which is believed to be involved in receptor binding and S2, believed to mediate membrane fusion between the virus and target cell.

The S (spike) protein may form non-covalently linked homotrimers (oligomers), which may mediate receptor binding and virus infectivity. Homotrimers of S proteins are likely necessary for presenting the correct native conformation of receptor binding domains and for eliciting a neutralizing antibody response. In addition, intracellular processing of S protein is associated with significant posttranslation oligosaccharide modification. The posttranslation oligosaccharide modification (glycosylation) expected by N-glycan motif analysis indicates that the S protein has as many as 23 sites for such modification. In addition, C-terminal cysteine residues may also participate in protein folding and preserving the native (functional) S protein conformation. The S protein of some coronaviruses {e.g. some strains of group II and III viruses) can be proteolytically processed near the center of the S protein by a trypsin-like protease in the Golgi apparatus or by extracellularly localized enzymes into to a linked polypeptide, containing an N-terminal SI and a C-terminal S2 polypeptide. Some members of the type II group of coronaviruses and group I viruses may not be so processed. Until the characterization of the SARS-associated viral agent as a coronavirus, the coronaviruses were divided into three groups on the basis of serological and genetic properties, which groups were referred to as Group 1, Group 2, and Group 3, which are also referred to in the art and herein as Group I, Group II, and Group III (see e.g. Holmes, Fields Virology, supra; Stadler, Nat. Rev. Microbiol. (2003) 209-18; Holmes, J Clin. Invest. (2003) 111:1605-609). Presently, the coronaviruses are subdivided into Group 1, Group 2, Group 3, and SARS-CoV (SARS-associated coronavirus including SARS-CoV-2).

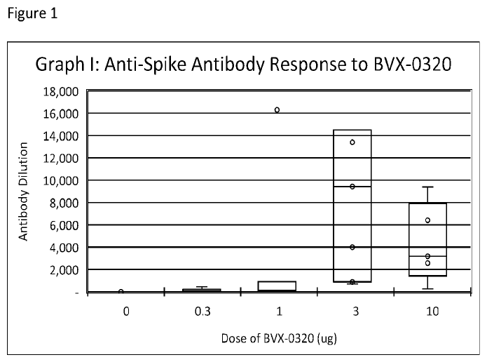

An exemplary S protein has 1,255 amino acids (see FIG. 1 which is SEQ ID NO:2), with a 12 amino acid signal sequence, the SI domain between amino acids 12-672 and the S2 domain between amino acids 673-1192. In certain embodiments, coronavirus S polypeptides and variants thereof that have one or more epitopes (i.e. are immunogens) and that are capable of eliciting a neutralizing (e.g. IgA or IgG antibody) or cell-mediated immune response, are included in compositions for use in treating or preventing coronavirus infections. Also described herein is the identification of S protein immunogens (containing one or more immunogenic epitopes) that are not glycosylated and that are capable of eliciting a neutralizing immune response. In one embodiment, the S protein immunogen is a portion or fragment of the full-length S protein. For example, a portion of the S protein immunogen that includes amino acids at positions 417-560 of SEQ ID NO:2 does not contain an N-glycan substitution site and is a hydrophilic region. This region also corresponds to the region of the 51 domain that is believed to be involved with cell receptor binding. Accordingly, a fragment comprising amino acids at positions 417-560 of SEQ ID NO:2, or a portion thereof, may be immunogenic and an immune response specific for one or more epitopes within this sequence may prevent entry of the coronavirus into a target cell. In addition, identification of such immunogenic fragments of the S protein that do not contain glycosylation sites provides the advantage that the fragments may be made and produced in cells, such as bacteria, that are not capable of glycosylating a protein in the same manner as a mammalian cell.

As described herein, an S protein immunogen includes a fragment of S protein or a S protein variant (which may be a variant of a full-length S protein or S fragment as described herein) that retains or that has at least one epitope contained within the full-length S protein or wildtype S protein, respectively, that elicits a protective immune response against coronavirus, preferably against SARS coronavirus. An S protein fragment or an S protein variant has at least one biological activity or function of a full-length or wildtype (natural) S protein (such as receptor binding or cell fusion activity), or has multiple S protein-specific biological activities or functions. For example, an S protein variant may contain an epitope that induces an immune response (for example, induces production of an antibody that specifically binds to a wildtype or full-length S polypeptide) or may have S protein receptor binding activity. In one embodiment, an S-protein fragment is a truncated S-protein that comprises an amino acid set forth at positions 1-1200 of SEQ ID NO:2. The portion of the S-protein that is deleted is the transmembrane region. S protein immunogenic fragments also include smaller portions or fragments of the aforementioned amino acid fragments of an S protein. An S protein fragment that comprises an epitope that stimulates, induces, or elicits an immune response may comprise a sequence of consecutive amino acids ranging from any number of amino acids between 8 amino acids and 150 amino acids (e.g. 8, 10, 12, 15, 18, 20, 25, 30, 35, 40, 50 or more amino acids) of SEQ ID NO:2. In related embodiments, a coronavirus S polypeptide variant has at least 85% to 100% amino acid sequence identity (that is, at least 85%, 90%, 95% or 99% sequence identity) to the amino acid sequence of the full length S protein as set forth in SEQ ID NO:2 (which is from SARS-CoV-2 strain; SEQ ID NO: 1 is the nucleic acid sequence that encodes the amino acid sequence of SEQ ID NO:2). Such S protein variants and fragments retain at least one S protein-specific biological activity or function, such as (1) the capability to elicit a protective immune response (that is, the S polypeptide variant contains an epitope that induces or elicits a protective immune response), for example, a neutralizing response and/or a cell-mediated immune response against coronavirus, such as SARS-CoV-2; (2) the capability to mediate viral infection via receptor binding; and (3) the capability to mediate membrane fusion between a virion and the host cell. Additional examples of full-length SARS coronavirus S (spike) polypeptide sequences are available in the art.

As described herein, S protein immunogens, fragments, and variants thereof described herein contain a haptenized epitope that elicits or induces an immune response, preferably a protective immune response, which may be a humoral response and/or a cell-mediated immune response. A protective immune response may be manifested by at least one of the following: preventing infection of a host by a coronaviras; modifying or limiting the infection; aiding, improving, enhancing, or stimulating recovery of the host from infection; and generating immunological memory that will prevent or limit a subsequent infection by a coronavirus. A humoral response may include production of antibodies that neutralize infectivity, lyse the virus and/or infected cell, facilitate removal of the virus by host cells (for example, facilitate phagocytosis), and bind to and facilitate removal of viral antigenic material. A humoral response may also include a mucosal response, which comprises eliciting or inducing a specific mucosal IgA response.

Induction of an immune response in a subject or host (human or non-human animal) by a haptenized S protein, fragment, or variant described herein, may be determined and characterized by methods described herein and routinely practiced in the art. These methods include in vivo assays, such as animal immunization studies (e.g. using a rabbit, mouse or rhesus macaque model), and any one of a number of in vitro assays, such as immunochemistry methods for detection and analysis of antibodies, including Western immunoblot analysis, ELISA, immunoprecipitation, radioimmunoassay, and the like, and combinations thereof. By way of example, animal models may be used for determining the capability of a coronavirus antigen to elicit and induce an immune response that is protective in animals, which may be determined by endpoints relevant to the particular model.

Other methods and techniques that may be used to analyze and characterize an immune response include neutralization assays (such as a plaque reduction assay or an assay that measures cytopathic effect (CPE) or any other neutralization assay practiced by persons skilled in the art) to assess whether a haptenized S protein immunogen or variant thereof is capable of eliciting an immune response, particularly a neutralizing immune response

The haptenized S protein immunogens (full-length proteins, variants, fragments, and fusion proteins thereof) are provided in an isolated form, and in certain embodiments, are purified to homogeneity. As used herein, the term “isolated” means that the polypeptide is removed from its original or natural environment.

In an embodiment, the invention provides a method of immunizing a human subject against a Coronavirus, the method comprising administering an effective amount of an haptenized S protein from Coronavirus, including SARS-CoV-2 Coronavirus. In some embodiments, the S protein is from SARS-CoV-2 Coronavirus and has the amino acid sequence as set forth in FIG. 1.

In some embodiments, the haptenized S protein is administered every other week for at least eight weeks. In some embodiments, the haptenized S protein is administered once per week for at least six weeks. In some embodiments, the method further comprising at least one booster injection of the haptenized S protein about six months after the first injection. In some embodiments, booster injections continue every six months or until an immunogenic response against Coronavirus occurs in the human subject.

In some embodiments, the effective amount of haptenized S protein is administered every other week until the delayed type hypersensitivity diagnostic test is positive, or a neutralizing antibody response is detected (e.g. anti-S protein antibodies are detected in the blood or serum of the human subject).

In an embodiment, the invention provides a method of producing an immunogenic response in a human subject against a Coronavirus, including SARS-CoV-2 Coronavirus, the method comprising administering an effective amount of an haptenized S protein from Coronavirus, including SARS-CoV-2 Coronavirus. In some embodiments, the S protein is from SARS-CoV-2 Coronavirus and has the amino acid sequence as set forth in FIG. 1 (SEQ ID NO:2).

### Methods of Immunization

Also described herein are methods for treating and/or preventing a coronavirus infection, comprising administering to a subject in need thereof a composition comprising at least one haptenized coronavirus S protein immunogen, wherein the S protein immunogen comprises an amino acid sequence that is identical to, or at least 85% identical to (which includes at least 90% or 95% or any percent in between 85% and 100%) SEQ ID NO: 2 and wherein the haptenized S protein immunogen has an epitope that elicits a protective immune response, which is a humoral immune response (including, for example, a mucosal IgA, systemic IgA, IgG, IgM response) and/or a cell-mediated immune response, and pharmaceutically acceptable carrier, diluent, or excipient. The haptenized S protein immunogen composition is administered at a dose sufficient to elicit an immune response specific for the administered haptenized S protein immunogen or immunogens or variants thereof. In certain embodiments, an infection being prevented or treated may be caused by a group 1 coronavirus, group 2 coronavirus, group 3 coronavirus, SARS group coronavirus (including SARS-CoV-2), or a combination thereof.

A human subject or host suitable for treatment with a coronavirus immunogen composition or formulation may be identified by well-established indicators of risk for developing a disease such as Covid-19 or by well-established hallmarks of an existing coronavirus disease. For example, indicators of an infection include fever, dry cough, dyspnea (shortness of breath), headache, hypoxaemia (low blood oxygen concentration), lymphopaenia (reduced lymphocyte numbers), mildly elevated aminotransferase levels (indicating liver damage), microorganism positive cultures, inflammation, and the like. Infections that may be treated or prevented with a haptenized coronavirus S protein immunogen vaccine as described herein include those caused by or due to coronavirus, whether the infection is primary, secondary or opportunistic. Examples of coronavirus include any subtype, strain, antigenic variant, and the like, of these viruses, including SARS coronavirus such as SARS-CoV-2. By way of example, SARS infections are characterized by flu-like symptoms, including high fever, myalgia, dry and non-productive dyspnea, lymphopenia, and infiltrate on chest radiography.

The immunogenic compositions that contain one or more haptenized coronavirus S protein immunogens of the invention may be in any form that allows for the composition to be administered to a subject, such as a human or non-human animal. For example, a haptenized S protein immunogen may be prepared and administered as a liquid solution or prepared as a solid form (e.g. lyophilized), which may be administered in solid form, or resuspended in a solution in conjunction with administration. The hybrid polypeptide composition is prepared or formulated to allow the active ingredients contained therein to be bioavailable upon administration of the composition to a subject or patient or to be bioavailable via slow release. Compositions that will be administered to a subject or patient take the form of one or more dosage units; for example, a tablet may be a single dosage unit, and a container of one or more compounds of the invention in aerosol form may hold a plurality of dosage units. In certain preferred embodiments, any of the aforementioned immunogenic compositions or vaccines comprising a haptenized coronavirus S Protein immunogen of the invention are in a container, preferably in a sterile container.

In one embodiment, the immunogenic composition or vaccine is administered nasally, wherein a haptenized coronavirus S protein immunogen can be taken up by cells, such as cells located in the nasal-associated lymphoid tissue. Other typical routes of administration include, without limitation, parenteral, transdermal/transmucosal, nasal, and inhalation. The term “parenteral” as used herein, describes administration routes that bypass the gastrointestinal tract, including intraarterial, intradermal, intramuscular, intranasal, intraocular, intraperitoneal, intravenous, subcutaneous, submucosal, and intravaginal injection or infusion techniques. The term “transdermal/transmucosal” as used herein, is a route of administration in which the immunogenic composition is administered through or by way of the skin, including topical. The terms “nasal” and “inhalation” encompass techniques of administration in which an immunogenic composition is introduced into the pulmonary tree, including intrapulmonary or transpulmonary. In one embodiment, the compositions of the present invention are administered nasally.

In some embodiments, the immunogenic composition or vaccine contains an amount of haptenized coronavirus S protein from about 60 μg to about 240 μg per dose. In some embodiments, the amount of haptenized coronavirus S protein administered per dose is from about 1 μg to about 240 μg. In some embodiments, the amount of haptenized S protein administered per dose is from about 1 μg, 3 μg, 5 μg, 10 μg, 25 μg, 30 μg, 40 μg, 50 μg, 60 μg, 70 μg, 80 μg, 90 μg, 100 μg, 110 μg, 120 μg, 130 μg, 140 μg, 150 μg, 160 μg, 170 μg, 180 μg, 190 μg, 200 μg, 210 μg, 220 μg, 230 μg, 240 μg or more. In some embodiments, the amount of haptenized coronavirus S protein varies depending on dosing schedule. For example, an initial dose may be the same as, lower or higher than any subsequent immunization dose, including a booster immunization dose. The amount of haptenized S protein administered per dose to any human subject can be adjusted according to the age, weight and physical condition of the human subject.

### Adjuvants

In some embodiments, the invention provides an immunogenic composition or vaccine comprising an haptenized S protein for injection further comprising a vaccine adjuvant.

In one embodiment, a composition that is useful as an immunogenic composition for treating and/or preventing a coronavirus infection contains at least one coronavirus antigen (immunogen) as described herein capable of eliciting an immune response and protollin or proteosome adjuvant (see e.g. U.S. Pat. No. 5,726,292). As is understood in the art, an adjuvant may enhance or improve the immunogenicity of an immunogen (that is, act as an immunostimulant), and many antigens are poorly immunogenic unless combined or admixed or mixed with an adjuvant. A variety of sources can be used as a source of antigen, such as live attenuated virus, killed virus, split antigen preparations, subunit antigens, recombinant or synthetic viral antigens, and combinations thereof. To maximize the effectiveness of a subunit, recombinant, or synthetic vaccine, the antigens can be combined with a potent immunostimulant or adjuvant. Other exemplary adjuvants include alum (aluminum hydroxide, REHYDRAGEL); aluminum phosphate; virosomes; liposomes with and without Lipid A; or other oil in water emulsions type adjuvants such as MF-59 (Novartis), also such as nanoemulsions (see e.g. U.S. Pat. No. 5,716,637) or submicron emulsions (see e.g. U.S. Pat. No. 5,961,970); and Freund's complete and incomplete adjuvant.

A proteosome-based adjuvant (i.e. protollin or proteosome) can be used in vaccine compositions or formulations that may include any one or more of a variety of coronavirus antigen (immunogen) sources as described herein. Proteosomes are comprised of outer membrane proteins (OMP) from Neisseria species typically, but can be derived from other Gram-negative bacteria (see e.g. U.S. Pat. No. 5,726,292). Proteosomes have the capability to auto-assemble into vesicle or vesicle-like OMP clusters of 20-800 nm, and to noncovalently incorporate, coordinate, associate, or otherwise cooperate with protein antigens, particularly antigens that have a hydrophobic moiety. Proteosomes are hydrophobic, safe for human use, and comparable in size to certain viruses. By way of background, and not wishing to be bound by theory, mixing proteosomes with an antigen such as a protein antigen, provides a composition comprising non-covalent association or coordination between the antigen and Proteosomes, which association or coordination forms when solubilizing detergent is selectively removed or reduced in concentration, for example, by dialysis.

Any preparation method that results in the outer membrane protein component in vesicular or vesicle-like form, including molten globular-like OMP compositions of one or more OMP, is included within the scope of proteosome. In one embodiment, the proteosomes are from Neisseria species, and from Neisseria meningitidis. In certain other embodiments, proteosomes may be an adjuvant and an antigen delivery composition. In an embodiment, an immunogenic composition comprises one or more coronavirus antigens and an adjuvant, wherein the adjuvant comprises Projuvant or Protollin. As described herein, a coronavirus antigen may be isolated from the virus particles, a cell infected by the coronavirus, or from a recombinant source. In certain embodiments, an immunogenic composition further comprises a second immunostimulant, such as a liposaccharide. That is, the adjuvant may be prepared to include an additional immunostimulant. For example, the projuvant may be mixed with a liposaccharide to provide an OMP-LPS adjuvant. Thus, the OMP-LPS (protollin) adjuvant can be comprised of two components. The first component includes an outer membrane protein preparation of proteosomes (i.e. Projuvant) prepared from Gram-negative bacteria, such as Neisseria meningitidis, and the second component includes a preparation of liposaccharide. It is also contemplated that the second component may include lipids, glycolipids, glycoproteins, small molecules or the like, and combinations thereof. As described herein, the two components of an OMP-LPS adjuvant may be combined (admixed or formulated) at specific initial ratios to optimize interaction between the components, resulting in stable association and formulation of the components for use in the preparation of an immunogenic composition. The process generally involves the mixing of components in a selected detergent solution (e.g. Empigen BB, Triton x-100 or Mega-10) and then effecting complex formation of the OMP and LPS components while reducing the amount of detergent to a predetermined, preferred concentration by dialysis or by diafiltration/ultrafiltration methodologies. Mixing, co-precipitation, or lyophilization of the two components may also be used to effect an adequate and stable association, composition, or formulation. In one embodiment, an immunogenic composition comprises one or more coronavirus haptenized S protein antigens and an adjuvant, wherein the adjuvant comprises a projuvant (i.e. proteosome) and liposaccharide.

In an embodiment, the final liposaccharide content by weight as a percentage of the total proteosome protein can be in a range from about 1% to about 500%, also in range from about 10% to about 200%, or in a range from about 30% to about 150%. Another embodiment includes an adjuvant wherein the proteosomes are prepared from Neisseria meningitidis and the liposaccharide is prepared from Shigella flexneri or Plesiomonas shigelloides, and the final liposaccharide content is between 50% to 150% of the total Proteosome protein by weight. In another embodiment, proteosomes are prepared with endogenous lipooligosaccharide (LOS) content ranging from about 0.5% up to about 5% of total OMP. In another embodiment proteosomes have endogenous liposaccharide in a range from about 12% to about 25%, and in still another embodiment the endogenous liposaccharide is between about 15% and about 20% of total OMP. The instant disclosure also provides an immunogenic composition containing liposaccharide derived from any Gram-negative bacterial species, which may be from the same Gram-negative bacterial species that is the source of proteosomes or may be from a different bacterial species. In certain embodiments, the proteosome or protollin to coronavirus antigen ratio in the immunogenic composition is greater than 1:1, greater than 2:1, greater than 3:1 or greater than 4:1. In other embodiments, proteosome or protollin to haptenized coronavirus S protein antigen ratio in the immunogenic composition is about 1:1, 2:1, 3:1 or 4:1. The ratio can be 8:1 or higher. In other embodiments, the ratio of proteosome or protollin to haptenized coronavirus S protein antigen of the immunogenic composition ranges from about 1:1 to about 1:500, and is at least 1:5, at least 1:10, at least 1:20, at least 1:50, or at least 1:100, or at least 1:200. An advantage of protollin to haptenized coronavirus S protein antigen ratios ranging from 1:2 to 1:200 is that the amount of proteosome-based adjuvant can be reduced dramatically with no significant effect on the ability of a coronavirus antigen to elicit an immune response. In another embodiment, an immunogenic composition comprises one or more haptenized coronavirus S protein immunogens combined (admixed or formulated) with proteosome or protollin, wherein the S protein immunogen comprises an amino acid sequence that is identical to, or at least 85% identical (which includes at least 90% or 95% or any percent in between 85% and 100%) to SEQ ID NO:2 or fragment thereof and wherein the haptenized S protein immunogen or fragment thereof has an epitope that elicits a protective immune response against coronavirus infection. An exemplary haptenized S protein immunogen comprises an amino acid sequence as set forth in SEQ ID NO:2 or consisting of SEQ ID NO:2. In other embodiments, an S protein immunogen is a fragment of SEQ ID NO:2, which fragment comprises an amino acid sequence that is identical to, or at least 85% identical (which includes at least 90% or 95% or any percent in between 85% and 100%) to an amino acid selected from SEQ ID NO:2.

Alternatively, any haptenized S protein immunogen as described herein can be combined (admixed or formulated) in an immunogenic composition with a liposome. Preferably, liposomes that contain one or more coronavirus immunogens further comprise Deinococcus radiodurans lipids or α-galactosylphosphotidylglycerolalkylamine. The addition of such lipids in a liposome can enhance the efficacy of a coronavirus vaccine composition by increasing protective immunity. Haptenized coronavirus S protein immunogens of the present invention may further include a covalently attached hydrophobic portion. A hydrophobic portion may be, for example, an amino acid sequence or a lipid, as disclosed in U.S. Pat. No. 5,726,292. Naturally occurring coronavirus S protein and a recombinantly expressed S protein having the sequence set forth in SEQ ID NO:2 contains a hydrophobic transmembrane domain (from about amino acid 1195 to about 1240 of SEQ ID NO:2), which may function as a hydrophobic portion with an S protein immunogen fragment. In certain other embodiments, the haptenized S protein immunogen, may further contain a second amino acid sequence to form a fusion protein, wherein the second amino acid sequence is a tag, carrier, or enzyme, as described herein.

In other embodiments, immunogenic compositions may comprise (projuvant or protollin), or further comprise components (e.g. receptor ligands) capable of stimulating a host immune response by interacting with certain receptors (e.g. Toll-like receptors or “TLR”) produced by one or more host cells of a vaccine recipient. According to one embodiment, compositions comprising immunogenic epitopes of a coronavirus protein may contain polypeptide epitopes capable of interacting with Toll-like receptors, thereby promoting an innate immune response, which may or may not evoke a subsequent adaptive immune response.

An innate immune response is a nonspecific protective immune response that is not a specific antigen-dependent or antibody-dependent response (that is, does not involve clonal expansion of T cells and/or B cells) and may be elicited by any one of numerous antigens, immunogens, or coronaviruses described herein. An immunostimulatory composition described herein comprises proteosomes and liposaccharide (protollin), either one of which or both may elicit a nonspecific protective response. Without wishing to be bound by theory, one or more components of vaccine compositions or formulations disclosed herein may interact with Toll-like receptors associated with an innate or adaptive immune response of a vaccine recipient. One or more ligands that interact with and subsequently activate certain TLR have been identified, with the exception of TLR8 and TLR10. Certain outer membrane proteins of Neisseria meningitidis, for example OMP2 (also referred to as PorB), interact with TLR2, while LPS of most but not all Gram-negative bacteria interacts with TLR4. Accordingly, one activity of vaccine compositions or formulations described herein, which may contribute to a biological effect, includes activation of one or both of TLR2 and TLR4. Activation of other TLR (other than TLR2 and TLR4) may serve a similar function or further enhance the qualitative or quantitative profile of cytokines expressed, and may be associated with a host Th1/Th2 immune response. It is also contemplated that TLR ligands other than LPS and PorB may be used alone or in combination to activate TLR2 or TLR4. The qualitative or quantitative activation of one or more TLR is expected to elicit, effect, or influence a relative stimulation (balanced or unbalanced) of a Th1 or Th2 immune response, with or without a concomitant humoral antibody response. Ligands interacting with TLR other than TLR2 and TLR4 may also be used in vaccine compositions described herein. Such vaccine components may, alone or in combination, be used to influence the development of a host immune response sufficient to treat or protect from virus infection, as set forth herein.

Other components known to the art may be used in the compositions described herein. Some embodiments of the haptenized S protein immunogen may further comprise adjuvants, such as Bacillus Calmette-Guérin (BCG), cytokines (for non-limiting example, granulocyte-macrophage colony-stimulating (GM-CSF)), aluminum gels or aluminum salts, or other adjuvants known to the art to non-specifically stimulate immune response and enhance the efficacy of the immune response to the vaccine. In at least one preferred embodiment, the adjuvant is BCG Tice.

A haptenized S protein immunogenic composition or vaccine may further comprise preservatives known to the art, including without limitation, formaldehyde, antibiotics, monosodium glutamate, 2-phenoxyethanol, phenol, and benzethonium chloride. A haptenized S protein immunogenic composition or vaccine may further comprise sterile water for injection, balanced salt solutions for injections.

While some embodiments of the invention are shown and described herein, such embodiments are provided by way of example only and are not intended to otherwise limit the scope of the invention. Various alternatives to the described embodiments of the invention may be employed in practicing the invention. Therefore, the spirit and scope of the appended claims should not be limited to the description of the preferred versions contained herein.

The attention of the reader is directed to all papers and documents which are filed concurrently with this specification and which are open to public inspection with this specification, and the contents of all such papers and documents incorporated herein by reference. All the features disclosed in the specification (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1. Preparing Haptenized S Protein

The following procedures illustrate exemplary haptenization procedures.

DNP modification. Modification of the prepared cells with DNP or another hapten may be performed by known methods, e.g. by the method of Miller, J. Immunol. (1976) 117:151 which involves a thirty minute incubation of S protein with DNFB under sterile conditions, followed by washing with sterile saline or HBSS-HSA. For example about 100 mg of DNFB (Sigma Chemical) can be dissolved in about 0.5 ml of 70% ethanol. About 99.5 ml of PBS is added. The solution is stirred overnight in a 37° C. water bath. The shelf life of the solution is about four weeks. The S protein is suspended in Hanks balanced salt solution. About 0.1 ml DNFB solution is added to each sample of S protein and incubated for about thirty minutes at room temperature.

SA modification. Modification of the S protein with SA may be performed by known methods. For example, in one embodiment, sulfanilic acid (SA) is converted to a diazonium salt by adding a saturating amount of sodium nitrite. Ice-cold, sterile filtered (0.2 μm), 10% sodium nitrite solution is added, dropwise, to a SA solution of 100 mg of anhydrous SA dissolved in 10 ml of 0.1N NCI until saturation. (The saturation point corresponds approximately to a final concentration of a sulfanilic acid diazonium salt of about 40 mM). The SA diazonium salt solution is then sterile filtered (0.2 μm membrane), and diluted 1:8 (v/v) in HBSS (without HSA). If needed, the pH is adjusted to 7.2 by dropwise addition of 1N NaOH. The SA diazonium salt-HBSS solution is then sterilized by filtration (0.2 μm membrane). S protein is suspended in diazonium salt-HBSS solution. The mixture is incubated for five minutes at room temperature. After the five minute incubation period, the hapenization reaction is stopped by the addition of 0.5 ml of a 25% HAS-H BSS solution to the mixture.

### Example 2. Clinical Studies

This Example outlines clinical studies for immunization with haptenized S protein.

A human coronavirus vaccine, consisting of recombinant S protein from SARS-CoV-2 modified with the hapten, dinitrophenyl (DNP) was prepared as per Example 1.

A phase I trial of the haptenized vaccine in patients with Covid-19 is conducted, testing four dosage levels. The major endpoints are the presence of a neutralizing antibody response to DNP-modified, SA-modified, and unmodified S protein. Also, the progression of Covid-19 is also assessed. Subsequently, a phase II trial using the lowest dose that is found to be immunologically effective in the phase I trial is conducted.

### Example 3. Administration Schedule

The table below shows an exemplary administration schedule for haptenized S protein.

| Timing | Description |
|---|---|
| Week 1, Day 1 | First haptenized S protein dose |
| | administered with adjuvant |
| Week 2, Day 8 | Second haptenized S protein dose |
| | administered with adjuvant |
| Week 3, Day 15 | Third haptenized S protein does |
| | administered with adjuvant |
| Week 26 (optional booster dose) | Fourth haptenized S protein dose |
| | administered with adjuvant |

### Example 4. Preparation of SARS-COV-2 S Protein Modified with Dinitrophenyl (BVX-0320)

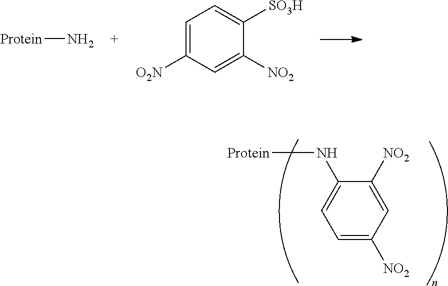

The following preparation is representative of the process used to prepare the haptenized S-Protein. The scale of the previous prep is given parenthetically. Filtration steps were added to produce a sterile product. Water should be low endotoxin water (nuclease free or WFI grade).
1. Prepare 0.1M sodium bicarbonate pH 8.2 buffer in water (nuclease free or WFI grade). Filter through 0.2-micron filter (Sigma s6014).
2. Thaw the requisite amount of S-protein (5 mg) (If necessary, filter through 0.2-micron filter).
3. Buffer exchange the S-protein with the bicarbonate buffer.
4. Determine concentration of S-protein (A280, 1.14 ml/mg*cm, target concentration 1 mg/mL).
5. Prepare 0.35M dinitrobenzene sulfonate (Sigma 556971) in 0.1M sodium bicarbonate buffer. Filter through 0.2-micron filter.
6. Combine S-protein (5 mL) and dinitrobenzene sulfonate (0.375 mL, 2000 equivalents) solutions in an appropriately sized vessel (original preparation split material into 10 vials) and stir gently for 18 hours at 30° C.
7. Ensure pH is greater or equal to 8.0
8. Purify reaction mixture using a Zeba column (equilibrated with 0.1M bicarbonate buffer) into PBS (pH 7.4). The purpose of this step is to both remove excess reagents as well as to perform the buffer exchange. The solution will be uncolored when all dinitrobenzene sulfonate is removed. Filter through 0.2-micron filter into sterile container.
9. Determine final concentration using BCA assay.
10. Analyze the final material for purity (SEC @ 280 nm), free hapten (RP-HPLC @ 235 nm), endotoxin by LAL, hapten loading (MS comparison with native protein).

### Example 5. Toxicity Study of BVX-0320 by Subcutaneous Injection in CF-1 Mice

CF-1 Mice (Charles River) were administered up to 10 μg of BVX-0320 by subcutaneous injection. Mortality was the an assessment of toxicity. All animals survived to the scheduled necropsy. There were no unusual BVX-0320-related clinical/veterinary observations. Additional clinical observations were within the range of normal findings for group-housed animals of this age, sex, and species, or were procedure-related and were not considered to be related to test article administration. Body Weight and Body Weight Gains. There were no BVX-0320-related body weight effects. Additional minor fluctuations among mean and individual body weight were considered sporadic, consistent with biologic variation, and/or negligible in magnitude and not related to test article administration. There were no BVX-0320-related food consumption effects. Minor fluctuations among mean and individual cage food consumption were considered sporadic, consistent with biologic variation, and/or negligible in magnitude and not related to test article administration. There were no unusual gross pathology findings.

In conclusion, there were no BVX-0320-related effects on body weight, food consumption or clinical signs. Based on these findings, BVX-0320 was determined to be well tolerated up to 10 μg/dose, the highest tested dose.

### Example 6. Immunology Study of BVX-0320 by Subcutaneous Injection in CF-1 Mice

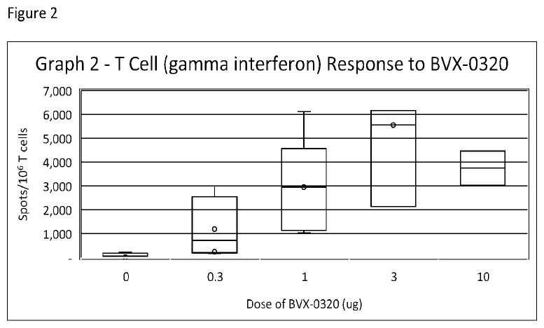

CF-1 Mice (Charles River) were administered 0.3, 1 or 3 μg of BVX-0320 by subcutaneous injection followed by a second injection and endpoints were obtained at six weeks (see FIGS. 1-2). For the 1 μg group: one mouse had baseline antibody titer 1:5. For the 3 μg group, the titre obtained was 1:120,000. Statistical analyses among the different groups yielded the following results: 0.3 μg group vs. 3 μg group, p=0.002, 1 μg group vs. 3 μg group, p=0.041, 3 μg group vs. 10 μg group, p=0.522.

Thus, two injections of BVX-0320 induced (1) Antibody against the S1 subunit of the spike protein of SARS-Cov-2 (FIG. 1). All 4 doses induced antibody, but titers were significantly higher in the two highest dose (3 μg, 10 μg groups). Adjuvant alone (dose=0 μg) did not induce an antibody response. Pre-vaccine sera were negative with the exception of a very low titer detected in a single sample. (2) Splenic T cells that produced the type I cytokine, gamma interferon, upon restimulation in vitro with a pool of peptides derived from the S1 subunit (FIG. 2). All four doses were effective. Adjuvant alone (dose=0 μg) did not induce a measurable response. (3) Splenic T cells, both CD4+ (Tables 2 and 4) and CD8+ (Tables 3 and 5) that became activated upon restimulation in vitro with the peptide pool. Activation was indicated by expression of CD25 (Tables 4 and 5) and CD69 (Tables 2 and 3). For three of the four parameters, only the two highest doses (3 μg, 10 μg) induced activation. Adjuvant alone (dose=0 μg) did not induce T cell activation. These results demonstrate that immunization with BVX-0320, a hapten (DNP)-modified S1 subunit of the spike protein, induced both antibody and T cell responses against the unmodified, native spike protein.

TABLE 2
Percent Activated Splenic CD4+, CD69+ T Cells After
Two Doses of BVX-0320 Cells were left unstimulated or stimulated
with a pool of peptides derived from the SARS-Cov-2 spike protein,
S1 subunit. The differences between unstimulated and peptide-
stimulated spleen cells were analyzed by paired t-test.
Dose = 0: QS21 only

| Unstim | Peptide |
|---|---|
| 19.3% | 19.5% |
| 21.2% | 22.2% |
| 35.8% | 39.9% |
| 10.4% | 10.3% |
| 9.0% | 10.6% |

p = NS
Dose = 3 μg

| Unstim | Peptide |
|---|---|
| 22.1% | 25.0% |
| 22.5% | 24.8% |
| 21.0% | 39.5% |
| 23.3% | 27.1% |
| 18.3% | 22.0% |
| 12.2% | 15.9% |
| 15.5% | 19.8% |

p = 0.021
Dose = 0.3 μg

| Unstim | Peptide |
|---|---|
| 17.6% | 16.5% |
| 23.4% | 25.4% |
| 30.1% | 23.7% |
| 20.2% | 33.2% |
| 19.1% | 19.5% |
| 25.2% | 33.3% |
| 16.5% | 21.5% |

p = NS
Dose = 10 μg

| Unstim | Peptide |
|---|---|
| 24.2% | 27.6% |
| 17.5% | 25.4% |
| 17.2% | 26.3% |
| 16.2% | 21.7% |
| 12.0% | 19.0% |
| 13.5% | 14.2% |

p = 0.004
Dose = 1 μg

| Unstim | Peptide |
|---|---|
| 13.4% | 14.3% |
| 15.6% | 16.1% |
| 19.6% | 29.6% |
| 14.1% | 28.1% |
| 17.8% | 17.5% |

p = NS

TABLE 3
Percent Activated Splenic CD8+, CD69+ T Cells After
Two Doses of BVX-0320 Cells were left unstimulated or stimulated
with a pool of peptides derived from the SARS-Cov-2 spike protein,
S1 subunit. The differences between unstimulated and peptide-
stimulated spleen cells were analyzed by paired t-test.
Dose = 0: QS21 only

| Unstim | Unstim |
|---|---|
| 26.9% | 24.1% |
| 38.5% | 29.5% |
| 63.3% | 38.7% |
| 18.6% | 44.1% |
| 9.8% | 20.5% |
| | 19.4% |
| | 27.1% |

Dose = 3 μg

| Unstim | Peptide |
|---|---|
| 24.1% | 27.9% |
| 29.5% | 31.9% |
| 38.7% | 48.0% |
| 44.1% | 46.1% |
| 20.5% | 21.0% |
| 19.4% | 20.1% |
| 27.1% | 26.7% |

p = 0.04
Dose = 0.3 μg

| Unstim | Peptide |
|---|---|
| 23.6% | 22.6% |
| 31.9% | 33.4% |
| 47.8% | 37.8% |
| 31.5% | 37.2% |
| 24.1% | 24.8% |
| 32.2% | 36.3% |
| 32.5% | 35.5% |

p = NS
Dose = 10 μg

| Unstim | Peptide |
|---|---|
| 24.8% | 27.1% |
| 27.3% | 33.4% |
| 20.9% | 27.3% |
| 20.6% | 28.2% |
| 22.7% | 27.0% |
| 17.7% | 19.1% |

p = 0.003
Dose = 1 μg

| Unstim | Peptide |
|---|---|
| 15.3% | 16.8% |
| 20.3% | 21.1% |
| 35.8% | 47.1% |
| 27.9% | 35.2% |
| 12.1% | 11.7% |

p = NS

TABLE 4
Percent Activated Splenic CD4+, CD25+ T Cells After
Two Doses of BVX-0320 Cells were left unstimulated or stimulated
with a pool of peptides derived from the SARS-Cov-2 spike protein,
S1 subunit. The differences between unstimulated and peptide-
stimulated spleen cells were analyzed by paired t-test.
Dose = 0 μg

| Unstim | Peptide |
|---|---|
| 4.7% | 4.1% |
| 3.5% | 3.3% |
| 2.3% | 2.2% |
| 1.9% | 1.9% |
| 3.1% | 2.8% |

p = NS
Dose = 3 μg

| Unstim | Peptide |
|---|---|
| 4.0% | 4.4% |
| 4.3% | 4.7% |
| 1.4% | 4.4% |
| 2.4% | 3.7% |
| 1.9% | 2.5% |
| 0.9% | 1.6% |
| 1.1% | 1.7% |

p = 0.016
Dose = 0.3 μg

| Unstim | Peptide |
|---|---|
| 3.7% | 2.9% |
| 2.8% | 2.3% |
| 2.5% | 3.6% |
| 1.1% | 1.9% |
| 3.1% | 3.2% |
| 1.4% | 2.2% |
| 1.3% | 2.5% |

p = NS
Dose = 10 μg

| Unstim | Peptide |
|---|---|
| 3.0% | 3.9% |
| 2.5% | 4.1% |
| 2.5% | 4.1% |
| 2.3% | 3.2% |
| 1.0% | 2.1% |
| 1.3% | 1.3% |

p = 0.005
Dose = 1 μg

| Unstim | Peptide |
|---|---|
| 2.5% | 2.5% |
| 4.1% | 4.0% |
| 1.9% | 4.0% |
| 1.2% | 4.1% |
| 0.9% | 0.8% |

p = NS

TABLE 5
Percent Activated Splenic CD8+, CD25+ T Cells After
Two Doses of BVX-0320 Cells were left unstimulated or stimulated
with a pool of peptides derived from the SARS-Cov-2 spike protein,
S1 subunit. The differences between unstimulated and peptide-
stimulated spleen cells were analyzed by paired t-test.
Dose = 0 μg

| Unstim | Peptide |
|---|---|
| 0.16% | 0.23% |
| 0.22% | 0.15% |
| 0.57% | 0.42% |
| 0.17% | 0.19% |
| 0.14% | 0.21% |

p = NS
Dose = 3 μg

| Unstim | Peptide |
|---|---|
| 0.19% | 0.46% |
| 0.17% | 0.27% |
| 0.06% | 0.78% |
| 0.00% | 0.22% |
| 0.04% | 0.21% |
| 0.12% | 0.18% |
| 0.27% | 0.18% |

p = 0.038
Dose = 0.3 μg

| Unstim | Peptide |
|---|---|
| 0.20% | 0.19% |
| 0.18% | 0.14% |
| 0.00% | 0.30% |
| 0.13% | 0.28% |
| 0.20% | 0.25% |
| 0.26% | 0.81% |
| 0.24% | 0.35% |

p = .044
Dose = 10 μg

| Unstim | Peptide |
|---|---|
| 0.20% | 0.36% |
| 0.28% | 0.33% |
| 0.35% | 0.33% |
| 0.18% | 0.35% |
| 0.21% | 0.36% |
| 0.09% | 0.17% |

p = 0.012
Dose = 1 μg

| Unstim | Peptide |
|---|---|
| 0.07% | 0.10% |
| 0.24% | 0.32% |
| 0.21% | 0.55% |
| 0.25% | 0.50% |
| 0.04% | 0.08% |

p = .038

Although the present invention has been described in connection with specific selected embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A immunogenic composition comprising an haptenized Spike protein (S protein) or fragment thereof from a coronavirus and at least one pharmaceutically acceptable carrier.

2. The immunogenic composition of claim 1, wherein the S protein is from SARS-CoV-2.

3. The immunogenic composition of claim 2, wherein the fragment thereof comprises the 51 domain of the S protein.

4. The immunogenic composition of claim 2, wherein the S protein comprises the amino acid sequence of SEQ ID NO:2.

5. The immunogenic composition of any preceding claim where haptenized S protein is present in an amount from 60 to 240 μg.

6. The immunogenic composition of any preceding claim wherein the hapten is dinitroflurobenzene (DNFB).

7. The immunogenic composition of any preceding claim wherein the hapten is selected from the group consisting of trinitrochlorobenzene (TNCB), 2,4-difluoronitrobenzene (DNFB), N-iodoacetyl-N′-(5-sulfonic-1-naphthyl)ethylenediamine (AED), sulfanilic acid (SA), trinitrophenol (TNP), and 2,4,6-trinitrobenzenesulfonic acid (TNBS).

8. The immunogenic composition of any preceding claim further comprising an adjuvant.

9. The immunogenic composition of claim 8, wherein the adjuvant is an oil in water emulsion.

10. The immunogenic composition of claim 9, wherein the oil is squalene oil.

11. The immunogenic composition of claim 10, wherein the adjuvant is M59.

12. A method of immunizing a human subject against a coronavirus comprising administering to the human subject the immunogenic composition of any of claims 1-11.

13. The method of claim 12, wherein the immunogenic composition is administered once per week for at least three weeks.

14. The method of claim 13, the method further comprising administering at least one booster injection of the immunogenic composition about twelve months after the first injection.

15. The method of any of claims 12-14, wherein the immunogenic composition is administered until a coronavirus neutralizing antibody response is detected in the human subject.

16. The method of any of claims 12-15, wherein the human subject is suffering from Covid-19.

17. The method of any of claims 12-15, wherein the human subject is not suffering from Covid-19.
